(19) United States
(12) Patent Application Publication
(10) Pub. No.: US 2020/0278353 A1
(43) Pub. Date: Sep. 3, 2020

(54) Protein Expression Analysis For Breast Cancer Prognosis And Treatment

(71) Applicant: NantOmics, LLC, Culver City, CA (US)

(72) Inventors: Sarit SCHWARTZ, Rockville, MD (US); Fabiola CECCHI, Potomac, MD (US); Yuan TIAN, Culver City, CA (US); Christina YAU, San Francisco, CA (US); Christopher SZETO, Culver City, CA (US); Todd HEMBROUGH, Gaithersburg, MD (US); Stephen Charles BENZ, Santa Cruz, CA (US)

(21) Appl. No.: 16/650,482

(22) PCT Filed: Sep. 26, 2018
(86) PCT No.: PCT/US2018/052981

§ 371 Date: Mar. 25, 2020

Related U.S. Application Data
(60) Provisional application No. 62/563,513, filed on Sep. 26, 2017.
(60) Provisional application No. 62/595,553, filed on Dec. 6, 2017.

(51) Int. Cl. G01N 33/68; A61K 31/704; A61P 35/00

(52) U.S. Cl. CPC G01N 33/6848; A61K 31/704; G01N 2800/7028; G01N 2800/52; A61P 35/00

(57) ABSTRACT

Methods are provided for identifying whether a cancer patient, and especially a breast cancer patient, will be responsive to treatment. Specified TOPO2A, IDO1 and/or p16 fragment peptides are precisely detected and quantitated by SRM-mass spectrometry directly in cancer cells collected from tumor tissue that was obtained from a cancer patient and compared to reference levels in order to determine if the cancer patient will positively respond to treatment. Measurement of TOPO2A provides a direct indication of whether a patient will respond to anthracycline-containing therapy, and, in particular, neoadjuvant anthracycline-containing therapy. Quantitative levels of IDO1 and p16 are compared to reference levels in order to determine if a breast cancer patient will likely demonstrate a pathologically complete response (pCR) of cancer after cancer therapy treatment, irrespective of the chosen treatment.

This application claims priority to U.S. provisional application Ser. Nos. 62/563,513 filed Sep. 26, 2017 and 62/595,553 filed on Dec. 6, 2017, the contents of each of which are hereby incorporated by reference in their entireties.

### INTRODUCTION

Methods are provided for treating cancer patients, and especially breast cancer patients, by assaying tumor tissue surgically-removed from patients and identifying those patients most likely to respond to treatment. Methods for identifying cancer patients, and in particular breast cancer patients, most likely to respond to treatment with anthracycline chemotherapy agents involve determining specific levels of TOPO2A directly in tumor cells derived from patient tumor tissue. Further methods involve measuring the level of protein expression of the IDO1 and p16 proteins in tumor tissue procured from a cancer patient, for example breast cancer, and provide prognostic information about likelihood of favorable outcome from cancer therapy, wherein the outcome is defined as a pathologically complete response (pCR) of the cancer in said patient. Expression levels of the TOPO2A, IDO1 and p16 proteins are determined by quantitating specified fragment peptides derived from subsequences of each of the full-length proteins in a Selected Reaction Monitoring/Multiple Reaction Monitoring (SRM/MRM) mass spectrometry assay. An SRM/MRM assay is used to detect and quantitate a specified fragment peptide in cells procured from cancer patient tissue, such as, for example formalin fixed breast cancer tissue.

The methods of measuring expression levels of the TOPO2A, IDO1 and/or p16 proteins in patient tumor tissue can be used for diagnosis of cancer, staging of the cancer, prognosis of cancer progression, likelihood of response to cancer treatment, demonstration of a pathologically complete response (pCR) of the cancer, and the like.

### BRIEF DESCRIPTION OF THE DRAWINGS

FIG. 1 shows p16 protein expression distribution (left panel) in breast cancer patients regardless of treatment (n=453). Patients whose tumor expressed p16 (>100 amol/ug) had higher pCR rates (right panel) than patients with lower p16 expression (OR: 4.51, Fisher's exact test p=1.51×10\hat{ }−10). The difference retained statistical significance in a logistic regression model adjusting for HR and HER2 status (OR: 2.65, p-value 0.00022).

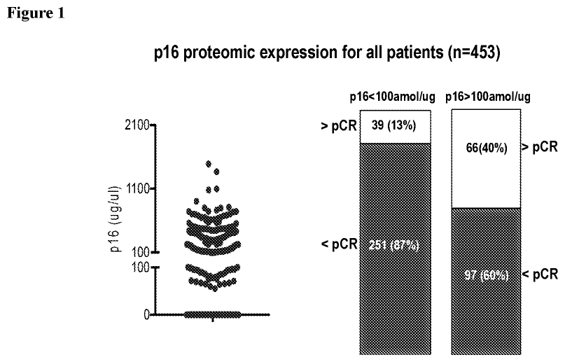

FIG. 2 shows IDO1 protein expression distribution (left panel) in breast cancer patients regardless of treatment (n=453). Patients whose tumor expressed IDO1 (>150amol/ug) had higher pCR rates (right panel) than patients with lower IDO1 expression (OR: 5.11, Fisher's exact test p=9.94×10\hat{ }−11). The difference retained statistical significance in a logistic regression model adjusting for HR and HER2 status (OR: 2.59, p-value 0.00076).

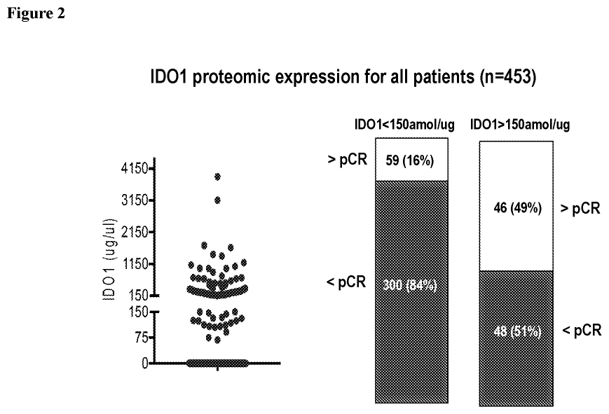

### SUMMARY OF THE INVENTION

Methods are provided for treating a patient suffering from cancer, such as breast cancer, by quantifying the level of TOPO2A, IDO1 and/or p16 fragment peptides in a protein digest prepared from a tumor sample obtained from the patient. The peptide(s) may be quantified by selected reaction monitoring using mass spectrometry by comparing the level of the TOPO2A, IDO1 and/or p16 fragment peptides to a reference level that is defined for each peptide. The measured levels of the peptide are then compared to corresponding reference levels, and the patient is treated with a therapeutic regimen comprising an effective amount of an anthracycline agent when (i) the level of TOPO2A is above the TOPO2A reference level, or (ii) the level of the IDO1 fragment peptide is above the IDO1 reference level, and/or when the level of the p16 fragment peptide is above the p16 reference level. When the TOPO2A, IDO1 and/or p16 levels are below the respective levels the patient is treated with an alternative therapeutic regimen. The protein digest may include a protease digest such as a trypsin digest. The specified TOPO2A peptide may have the amino acid sequence as set forth as SEQ ID NO:1, the specified IDO1 peptide may have the amino acid sequence as set forth as SEQ ID NO:2 and the specified p16 peptide may have the amino acid sequence as set forth as SEQ ID NO:3.

The tumor sample may be a cell, collection of cells, or a solid tissue, such as formalin fixed solid tissue, and/or paraffin embedded tissue.

The mode of mass spectrometry may be, for example, tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry and may be carried out using Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

In these methods quantifying the specified TOPO2A, IDO1 and p16 fragment peptides may be achieved, for example, by comparing to a spiked internal standard peptide of known amount, where both the native peptide in the biological sample and the internal standard peptide corresponds to the same amino acid sequence of the TOPO2A, IDO1 and p16 fragment peptides shown in SEQ ID NOs:1, 2, and 3 respectively. In each case the internal standard peptide may be an isotopically labeled peptide, such as a peptide labeled with one or more heavy stable isotopes selected from 18O, ¹⁷O, ¹⁵N, ¹³C, ²H or combinations thereof.

In the methods described herein the specified level of the TOPO2A peptide fragment may be 515±150 or 515±100 or 515±50 or 515±25 amol/ug protein analyzed, the specified level of the IDO1 peptide fragment may be 150±25 or 150±50 amol/ug protein analyzed, and/or the specified level of the p16 peptide fragment may be 100±50 or 100±25 amol/ug protein analyzed.

Detecting and quantitating the specified TOPO2A, IDO1 and p16 fragment peptides can be combined with detecting and quantitating other peptides from other proteins in multiplex so that the treatment decision about which agent used for treatment is based upon specific levels of the specified fragment peptide(s) in combination with other peptides/proteins in the biological sample.

### DETAILED DESCRIPTION

Methods are provided for determining if a cancer patient, and specifically a breast cancer patient, will clinically respond in a favorable manner to treatment. Measurement of IDO1 and p16 allows determination of whether a patient will respond to any therapy, and measurement of TOPO2A allows determination of whether a patient will respond to anthracycline-based therapy in particular.

The IDO1 (Indoleamine 2,3-dioxygenase, IDO) protein is a 403 amino acid enzyme that catalyzes the degradation of the essential amino acid L-tryptophan to N-formylkynurenine. IDO1 is an immune checkpoint molecule because tryptophan shortage inhibits division of T-lymphocytes. A wide range of human cancers such as prostatic, colorectal, pancreatic, cervical, gastric, ovarian, head, and lung cancer overexpress IDO1. The net effect of this overexpression by tumor cells likely is suppression of the immune system which improve the tumor cells' chances for avoiding immune surveillance and killing by the immune system. Accordingly, higher levels of the IDO1 protein in tumor cells indicate a less favorable outlook for a successful treatment response, while lower levels indicate a more favorable possibility that treatment would be successful.

The p16 protein (cyclin-dependent kinase inhibitor 2A, multiple tumor suppressor 1, CDKN2A) binds to the cyclin D-cyclin-dependent kinase 4 and 6 (CDK4/6) protein complex and inactivates it, resulting in cell-cycle arrest. In contrast, the loss of p16 protein expression through homozygous gene deletion, promoter methylation (transcription suppression), or inactivating point mutations can drive tumor growth through deregulated activation of the CDK4/6 protein complex. Loss of p16 protein expression most likely leads to deregulated tumor growth and indicates an unfavorable outlook for the future well-being of the patient, while increased p16 expression most likely slows tumor growth and provides a more favorable outlook for patient treatment response. In addition, loss of p16 expression may indicate that treating the patient with inhibitors of CDK4/6 pathway proteins may have a positive effect on treatment outcome for the patient.

The anthracycline class of chemotherapy agents includes epirubicin, doxorubicin (Adriamycin), daunorubicin and idarubin. Anthracyclines interact with DNA by intercalation thus inhibiting the progression of the enzyme topoisomerase II (TOPO2A) which relaxes supercoils in DNA for transcription. Anthracyclines stabilizes the TOPO2A complex after it has broken the DNA chain for replication, preventing the DNA double helix from being resealed and thereby stopping the process of replication which results in preventing cancer cells from synthesizing DNA and thus stopping cancer cell division and tumor growth. However, high expression levels of the TOPO2A enzyme in cancer cells can overcome the effects of anthracyclines and thus provide resistance to the effects of the drugs in cancer cells allowing them to synthesize DNA and thus promote cellular division and tumor growth.

TOPO2A is an enzyme that is a critical component of the DNA synthesis cellular process. It controls and alters the topologic states of DNA during transcription, by catalyzing the transient breaking and rejoining of two strands of duplex DNA which allows the strands to pass through one another, thus altering the topology of DNA. Studies of the relationship between tumor expression of TOPO2A protein and response to anthracycline-based chemotherapy have yielded contradictory results. The methods described herein use mass spectrometry (MS) to evaluate associations between tumor molecular profiles and pathological complete response (pCR) in breast cancer patients treated with anthracycline-containing therapy and, in particular, neoadjuvant anthracycline-containing therapy. The quantitative level of TOPO2A protein expression in patient tumor cells as determined by SRM/MRM is compared to a defined cutoff and, if TOPO2A is expressed at a level higher than a defined cutoff then the patient is surprisingly likely to respond to anthracycline-based therapy. Similarly, measurement of IDO1 and p16 levels in tumor cells can be compared to predetermined cutoffs and the resulting comparisons used to inform treatment decisions, as described in more detail below.

With respect to measurement of TOPO2A, diagnostic methods for measuring the TOPO2A protein in a tumor sample or samples from the patient are provided. The sample is advantageously formalin-fixed. Using an SRM/MRM assay that measures a TOPO2A peptide fragment, and particular characteristics about the peptide, the amount of the TOPO2A protein in cells derived from formalin fixed paraffin embedded (FFPE) tissue is determined. The peptide fragment is derived from the full-length TOPO2A protein, where the peptide sequence for the TOPO2A protein fragment is SEQ ID NO:1 (TLAVSGLGVVGR). Surprisingly it has been found that this peptide can be reliably detected and quantitated simultaneously in digests prepared from FFPE samples of tumor tissue. See U.S. patent application Ser. No. 13/993,045, the contents of which are hereby incorporated by reference in their entirety.

With respect to measurement of IDO1 and p16, the methods described herein provide a prognostic indicator for breast cancer that will indicate the likelihood of a pathologically complete response (pCR) for breast cancer, irrespective of the chosen treatment. This method is based on quantitative proteomics-based assays that quantify IDO1 and p16 proteins directly in formalin fixed tissues from cancer patients. Data from these assays can be used to predict a pCR to cancer therapy treatment for breast cancer patients and can be used to make improved treatment decisions for cancer therapy. In particular, the methods described herein can be used predict a pCR to therapy with an anthracycline agent such as daunorubicin, doxorubicin, or epirubicin. Also provided are improved methods of treatment wherein IDO1 and p16 are quantitated in patient tumor tissue and compared to a predetermined reference levels. When the level of the IDO1 fragment peptide is above the IDO1 reference level and/or when the level of the p16 fragment peptide is above the p16 reference level then the patient is treated with an anthracycline agent such as daunorubicin, doxorubicin, or epirubicin. Alternatively, if the level of the IDO1 fragment peptide is below the IDO1 reference level and/or the level of the p16 fragment peptide is below the p16 reference level the patient is treated with an alternative therapeutic regimen that does not include an effective amount of an anthracycline agent. Such an alternative regimen could include surgery such as lumpectomy or mastectomy, radiation treatment, or treatment with a chemotherapeutic agent such as Capecitabine (Xeloda), Carboplatin (Paraplatin), Cisplatin (Platinol), Cyclophosphamide (Neosar), Docetaxel (Docefrez, Taxotere), Fluorouracil (5-FU, Adrucil), Gemcitabine (Gemzar), Methotrexate, Paclitaxel (Taxol), Protein-bound paclitaxel (Abraxane), Vinorelbine (Navelbine), Eribulin (Halaven) and/or Ixabepilone (Ixempra). Therapeutic regimens using trastuzumab may also be used.

The peptides present in Table 1 are useful for measuring levels of the TOPO2A, IDO1 and p16 proteins in a complex Liquid Tissue lysate prepared from cells procured from formalin fixed cancer tissue. Unless noted otherwise, in each instance the protease is trypsin.

TABLE 1

| SEQ ID NO | Protein | Peptide Sequence |
|---|---|---|
| SEQ ID NO: 1 | TOPO2A | TLAVSGLGVVGR |
| SEQ ID NO: 2 | IDO1 | HLPDLIESGQLR |
| SEQ ID NO: 3 | P16 | ALLEAGALPNAPNSYGR |

More specifically, an SRM/MRM assay can measure one or more of these peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus, molecular analytical methods to analyze formalin fixed cancer tissue will be the most accepted and heavily utilized methods for analysis of cancer patient tissue.

The assays described herein measure relative or absolute levels of specific fragment peptides from the specified proteins. Relative quantitative levels of each of the proteins are determined by the SRM/MRM methodology for example by comparing SRM/MRM signature peak areas (e.g., signature peak area or integrated fragment ion intensity) of an individual fragment peptide derived from a protein in different samples. Alternatively, it is possible to compare multiple SRM/MRM signature peak areas for multiple signature peptides, where each peptide has its own specific SRM/MRM signature peak, to determine the relative protein content in one biological sample with the content of the same protein(s) in one or more additional or different biological samples. In this way, the amount of a particular peptide, or peptides, from the subject protein(s), and therefore the amount of the designated protein(s), is determined relative to the same peptide, or peptides, across 2 or more biological samples under the same experimental conditions. In addition, relative quantitation can be determined for a given peptide, or peptides, from a given protein within a single sample by comparing the signature peak area for that peptide by SRM/MRM methodology to the signature peak area for another and different peptide, or peptides, from a different protein, or proteins, within the same protein preparation from the biological sample. In this way, the amount of a particular peptide from a designated protein, and therefore the amount of that protein, is determined relative one to another within the same sample. These approaches generate quantitation of an individual peptide, or peptides, from a designated protein to the amount of another peptide, or peptides, between samples and within samples wherein the amounts as determined by signature peak area are relative one to another, regardless of the absolute weight to volume or weight to weight amounts of the selected peptide in the protein preparation from the biological sample. Relative quantitative data about individual signature peak areas between different samples are normalized to the amount of protein analyzed per sample. Relative quantitation can be performed across many peptides from multiple proteins and one or more of the designated proteins simultaneously in a single sample and/or across many samples to gain insight into relative protein amounts, such as one peptide/protein with respect to other peptides/proteins.

Absolute quantitative levels of the designated protein are determined by, for example, the SRM/MRM methodology whereby the SRM/MRM signature peak area of an individual peptide from the designated protein in one biological sample is compared to the SRM/MRM signature peak area of a spiked internal standard. In one embodiment, the internal standard is a synthetic version of the same exact peptide derived from the designated protein that contains one or more amino acid residues labeled with one or more heavy isotopes. Such isotope labeled internal standards are synthesized so that when analyzed by mass spectrometry a standard generates a predictable and consistent SRM/MRM signature peak that is different and distinct from the native peptide signature peak and which can be used as a comparator peak. Thus when the internal standard is spiked into a protein preparation from a biological sample in known amounts and analyzed by mass spectrometry, the SRM/MRM signature peak area of the native peptide is compared to the SRM/MRM signature peak area of the internal standard peptide, and this numerical comparison indicates either the absolute molarity and/or absolute weight of the native peptide present in the original protein preparation from the biological sample. Absolute quantitative data for fragment peptides are displayed according to the amount of protein analyzed per sample. Absolute quantitation can be performed across many peptides, and thus proteins, simultaneously in a single sample and/or across many samples to gain insight into absolute protein amounts in individual biological samples and in entire cohorts of individual samples.

Results from the SRM/MRM assay can be used to correlate accurate and precise quantitative levels of the TOPO2A, IDO1 and/or p16 proteins within the specific cancer of the patient from whom the tissue was collected and preserved, including breast cancer tissue. This not only provides diagnostic/prognostic information about the cancer, but also permits a physician or other medical professional to determine appropriate therapy for the patient. In this case, utilizing this assay can provide information about the specific level(s) of TOPO2A, IDO1 and/or p16 protein expression in cancer tissue and whether or not the patient from whom the cancer tissue was obtained will respond in a favorable way to anthracycline-based therapy.

Presently the most widely-used and applied methodology to determine protein presence in cancer patient tissue, especially FFPE tissue, is immunohistochemistry (IHC). IHC methodology utilizes an antibody to detect the protein of interest. The results of an IHC test are most often interpreted by a pathologist or histotechnologist. This interpretation is subjective and does not provide quantitative data that are predictive of sensitivity to therapeutic agents that target specific oncoprotein targets, such as anthracyclines, in a TOPO2A positive tumor cell population.

Research from other IHC assays, such as the Her2 IHC test, suggest the results obtained from such tests may be wrong. This is probably because different labs have different rules for classifying positive and negative IHC status. Each pathologist running the tests also may use different criteria to decide whether the results are positive or negative. In most cases, this happens when the test results are borderline, meaning that the results are neither strongly positive nor strongly negative. In other cases, tissue from one area of cancer tissue can test positive while tissue from a different area of the cancer tests negative. Inaccurate IHC test results may mean that patients diagnosed with cancer do not receive the best possible care. If all or part of a cancer is positive for a specific target oncoprotein but test results classify it as negative, physicians are unlikely to recommend the correct therapeutic treatment, even though the patient could potentially benefit from those agents. If a cancer is oncoprotein target negative but test results classify it as positive, physicians may recommend a specific therapeutic treatment, even though the patient is unlikely to get any benefits and is exposed to the agent's secondary risks. Accordingly, there is great clinical value in the ability to correctly evaluate the quantitative level of the TOPO2A, IDO1, and/or p16 proteins in tumors, especially breast tumors, so that the patient will have the greatest chance of receiving optimal treatment.

Quantitative levels of specified TOPO2A, IDO1 and/or p16 fragment peptides are determined in a mass spectrometer by the SRM/MRM methodology, whereby the SRM/MRM signature chromatographic peak area of the peptide is determined within a complex peptide mixture present in a Liquid Tissue lysate (see U.S. Pat. No. 7,473,532, as described above). Quantitative levels of the TOPO2A, IDO1 and/or p16 proteins are then determined by the SRM/MRM methodology whereby the SRM/MRM signature chromatographic peak area of one or more individual specified peptides from TOPO2A, IDO1 and/or p16 proteins in one biological sample is compared to the SRM/MRM signature chromatographic peak area of a known amount of a “spiked” internal standard for the same TOPO2A, IDO1 and/or p16 fragment peptides. In one embodiment, the internal standard is a synthetic version of the same exact TOPO2A, IDO1 and/or p16 fragment peptide where the synthetic peptide contains one or more amino acid residues labeled with one or more heavy isotopes. Such isotope labeled internal standards are synthesized so that mass spectrometry analysis generates a predictable and consistent SRM/MRM signature chromatographic peak that is different and distinct from the native TOPO2A, IDO1 and/or p16 fragment peptide chromatographic signature peak and which can be used as a comparator peak. Thus when the internal standard is spiked in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for a fragment peptide is displayed according to the amount of protein analyzed per sample.

In order to develop the SRM/MRM assay for the TOPO2A, IDO1 and/or p16 fragment peptides, additional information is utilized by the mass spectrometer. That additional information is used to direct and instruct the mass spectrometer, (e.g., a triple quadrupole mass spectrometer) to perform the correct and focused analysis of the specified TOPO2A, IDO1 and/or p16 fragment peptide. An SRM/MRM advantageously is performed on a triple quadrupole mass spectrometer. That type of a mass spectrometer may be considered to be the most suitable instrument for analyzing a single isolated target peptide within a very complex protein lysate that may consist of hundreds of thousands to millions of individual peptides from all the proteins contained within a cell. The additional information provides the triple quadrupole mass spectrometer with the correct directives to allow analysis of a single isolated target peptide within a very complex protein lysate that may consist of hundreds of thousands to millions of individual peptides from all the proteins contained within a cell. Although SRM/MRM assays can be developed and performed on any type of mass spectrometer, including a MALDI, ion trap, ion trap/quadrupole hybrid, or triple quadrupole, presently the most advantageous instrument platform for SRM/MRM assay is often considered to be a triple quadrupole instrument platform. The additional information about target peptides in general, and in particular about the specified TOPO2A, IDO1 and/or p16 fragment peptides, may include one or more of the mono isotopic mass of the peptide, its precursor charge state, the precursor m/z value, the m/z transition ions, and the ion type of each transition ion. The peptide sequences of the specified TOPO2A, IDO1 and/or p16 fragment peptides are shown in Table 1.

To determine an appropriate reference level for TOPO2A, IDO1 or p16 quantitation, tumor samples are obtained from a cohort of patients suffering from cancer, for example breast cancer. The tumor samples are formalin-fixed using standard methods and the level of TOPO2A, IDO1 and/or p16 in the samples is measured using the methods as described above. The tissue samples may also be examined using IHC and FISH using methods that are well known in the art. The patients in the cohort are treated with anthracycline-based therapy and the response of the patients is measured using methods that are well known in the art, for example by recording the overall survival or pathological complete response (pCR) of the patients at time intervals after treatment. A suitable reference level can be determined using statistical methods that are well known in the art, for example by determining the lowest p value of a log rank test.

Once a reference level has been determined it can be used to identify patients whose TOPO2A protein expression level indicates that they may likely benefit from anthracycline-based therapy. Levels of TOPO2A protein in patient tumor samples are typically expressed in amol/μg, although other units can be used. Similarly, once reference levels for IDO1 and p16 have been determined they can be used to identify patients whose IDO1 and p16 protein expression levels indicate how they will respond to therapy, in in particular anthracycline-based therapy. Levels of TOPO2A, IDO1 and p16 proteins in patient tumor samples are typically expressed in amol/μg, although other units can be used. The skilled artisan will recognize that a reference level can be expressed as a range around a central value, for example, +/−250, 150, 100, 50 or 25 amol/μg. In the methods described herein the specified level of the TOPO2A peptide fragment may be 515±150 or 515±100 or 515±50 or 515±25 amol/μg protein analyzed, the specified level of the IDO1 peptide fragment may be 150±25 or 150±50 amol/ug protein analyzed, and/or the specified level of the p16 peptide fragment may be 100±50 or 100±25 amol/ug protein analyzed.

Because both nucleic acids and protein can be analyzed from the same Liquid Tissue biomolecular preparation it is possible to generate additional information about disease diagnosis and drug treatment decisions from the nucleic acids in same sample upon which proteins were analyzed. For example, if the TOPO2A, IDO1 or p16 proteins are expressed by certain cells at increased levels, when assayed by SRM the data can provide information about the state of the cells and their potential for uncontrolled growth, choice of optimal therapy, and potential drug resistance. At the same time, information about the status of genes and/or the nucleic acids and proteins they encode (e.g., mRNA molecules and their expression levels or splice variations) can be obtained from nucleic acids present in the same Liquid Tissue biomolecular preparation. Nucleic acids can be assessed simultaneously to the SRM analysis of proteins, including the TOPO2A, IDO1 and/or p16 protein. In one embodiment, information about one or more of the TOPO2A, IDO1 and p16 proteins and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

### EXAMPLE 1: IDENTIFYING PATIENTS SENSITIVE TO ANTHRACYCLINE-CONTAINING THERAPY WITH QUANTITATIVE PROTEOMIC AND GENOMIC PROFILING

Background: Selecting chemotherapy based on tumor biology can improve response rates and avert toxicity. Studies of the relationship between tumor expression of TOPO2A protein and response to anthracycline-based chemotherapy have yielded contradictory results. Mass spectrometry (MS) was used to evaluate associations between tumor molecular profiles and pathological complete response (pCR) in breast cancer patients treated with neoadjuvant anthracycline-containing therapy.

Methods: Patients were selected from the ERNEST-B (Erlangen Neoadjuvant Study Breast), which is a retrospective cohort study. Archived tumor samples from anthracycline-treated breast cancer patients (n=133) were microdissected and solubilized. In each tumor sample, TOPO2A and other target proteins were quantitated with a mass spectrometry assay. Molecular profiling also included RNA sequencing of the tumor and whole genome sequencing of both tumor and matched normal tissue sections. The cohort was dichotomized into high and low expressors of TOPO2A using a protein level cutoff of 515 amol/μg of tumor protein. The difference in pCR (ypT0ypN0) rates between high and low expressors of TOPO2A was assessed using a z-test for differences in proportion.

Results: TOPO2A protein was detected in 84 of 133 (63%) tumor samples from anthracycline-treated patients (range: 178 to 3044 amol/μg). Patients whose tumor expressed TOPO2A protein above the cutoff of 515 amol/μg had higher pCR rates than patients with lower TOPO2A expression (35.5% vs. 12.1%; odds ratio (OR): 4.48 [95% CI: 1.53-13.28], Fisher Exact test p=0.004). The difference retained statistical significance in a logistic regression model adjusting for HR and HER2 status (OR: 2.23 [95% CI: 1.15-4.30], p=0.017). In a separate cohort of 19 breast cancer patients who did not receive anthracycline, there was no association between TOPO2A protein expression level and pCR rate.

Conclusions: In a retrospective analysis of anthracycline-treated breast tumors, TOPO2A expression was associated with a higher rate of pCR.

1. A method of treating a patient suffering from cancer, the method comprising:
(a) quantifying a level of a TOPO2A fragment peptide in a protein digest prepared from a tumor tissue sample obtained from the patient using mass spectrometry, and
(b) treating the patient with a therapeutic regimen comprising an effective amount of at least one anthracycline agent when the level of the TOPO2A fragment peptide is above a reference level, or
(c) treating the patient with a different therapeutic regimen when the level of the TOPO2A fragment peptide is below said reference level.

2. (canceled)

3. (canceled)

4. The method of claim 1, wherein said protein digest comprises a protease digest.

5. The method of claim 4, wherein said protein digest comprises a trypsin digest.

6. The method of claim 1, wherein said mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

7. The method of claim 1, wherein a mode of the mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

8. The method of claim 1, wherein the TOPO2A fragment peptide has the amino acid sequence as set forth as SEQ ID NO:1.

9. (canceled)

10. (canceled)

11. The method of claim 1, wherein the tumor sample is a cell, collection of cells, or a solid tissue.

12. The method of claim 11, wherein the tumor sample is formalin fixed solid tissue.

13. The method of claim 12, wherein the tissue is paraffin embedded tissue.

14. The method of claim 1, wherein quantifying the TOPO2A fragment peptide comprises determining the level of the TOPO2A fragment peptide in said sample by comparing to a spiked internal standard peptide of known amount, wherein both the TOPO2A fragment peptide in the sample and the internal standard peptide corresponds to the same amino acid sequence of the TOPO2A fragment peptide as shown in SEQ ID NO:1.

15. (canceled)

16. (canceled)

17. The method of claim 14, wherein the internal standard peptide is an isotopically labeled peptide.

18. The method of claim 17, wherein the isotopically labeled internal standard peptide comprises one or more heavy stable isotopes selected from ¹⁸O, ¹⁷O, ³⁴S, ¹⁵N, ¹³C, ²H and a combination thereof.

19. The method of claim 1, wherein the level of the TOPO2A fragment peptide is 515±150, 515±100, 515±50 or 515±25 amol/μg protein analyzed.

20. (canceled)

21. (canceled)

22. The method of claim 1, wherein detecting and quantitating the TOPO2A fragment peptide can be combined with detecting and quantitating other peptides from other proteins in multiplex format.

23. The method of claim 1, wherein said at least one anthracycline agent is selected from the group consisting of epirubicin, doxorubicin (Adriamycin), daunorubicin and idarubin.

24. The method of claim 1, wherein said patient is treated with neoadjuvant anthracycline-containing therapy.

25. (canceled)


SEQUENCE LISTING (3 sequences)

SEQ ID NO: 1 - protein, 12 aa, Homo sapiens
TLAVSGLGVVGR

SEQ ID NO: 2 - protein, 12 aa, Homo sapiens
HLPDLIESGQLR

SEQ ID NO: 3 - protein, 17 aa, Homo sapiens
ALLEAGALPNAPNSYGR